(19) United States
(12) Patent Application Publication
(10) Pub. No.: US 2011/0040091 A1
(43) Pub. Date: Feb. 17, 2011

(54) PROCESS FOR THE PREPARATION OF (R)-2-PHENYL PROPIONIC ACID DERIVATIVES

(76) Inventors: Stephan Bachmann, Allschwil (CH); Alec Fettes, Zuerich (CH); Michelangelo Scalone, Birsfelden (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/843,081

(22) Filed: Jul. 26, 2010

(30) Foreign Application Priority Data
Aug. 13, 2009 (EP) 09167753.4

(51) Int. Cl. C07D 241/20; C07C 315/00

(52) U.S. Cl. 544/336; 562/429

(57) ABSTRACT

The present invention relates to a process for the preparation of (R)-2-phenyl propionic acid derivatives of the formula

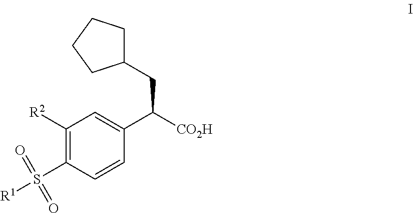

wherein R¹ is C₁₋₆-alkyl and R² is hydrogen or halogen, or of a salt thereof.

### PRIORITY TO RELATED APPLICATION(S)

This application claims the benefit of European Patent Application No. 09167753.4, filed Aug. 13, 2009, which is hereby incorporated by reference in its entirety.

### FIELD OF THE INVENTION

The present invention relates to a process for the preparation of (R)-2-phenyl propionic acid derivatives of the formula

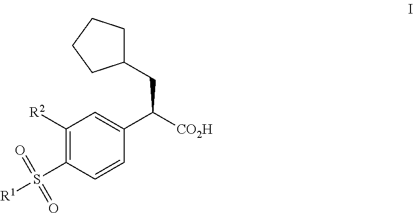

wherein R¹ is C₁₋₆-alkyl and R² is hydrogen or halogen, or of a salt thereof.
(R)-2-phenyl propionic acid derivatives of the formula I are key intermediates in the synthesis of 5-substituted-pyrazine or pyridine glucokinase activators of the formula Xa,

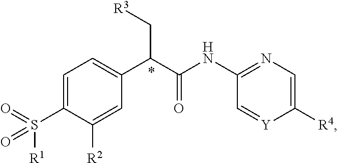

as disclosed in PCT International Patent Application No. WO 2004/052869 A1, for example 2-(3-chloro-4-methanesulfonyl-phenyl)-3-cyclopentyl-N-[5-(1,2-dihydroxy-ethyl)-pyrazin-2-yl]-propionamide, of the formula Xb,

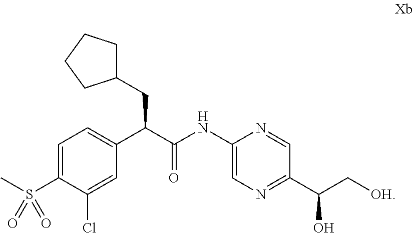

The glucokinase activators are useful for the treatment and/or prophylaxis of type II diabetes.

### SUMMARY OF THE INVENTION

The present invention provides process for the preparation of a (R)-2-phenyl propionic acid derivative of formula I,

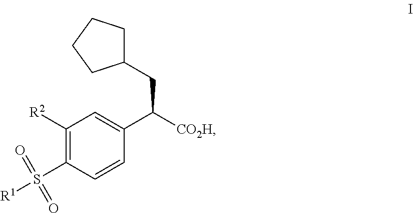

or a salt thereof.

### DETAILED DESCRIPTION OF THE INVENTION

The present invention relates to a process for the preparation of a (R)-2-phenyl propionic acid derivative of formula I,

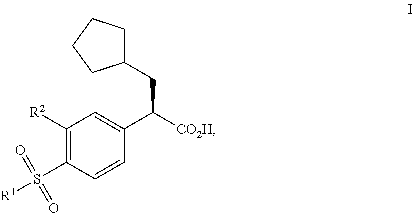

or a salt thereof.

The process comprises one or more of the following steps:

a) oxidation of a sulfide of formula II,

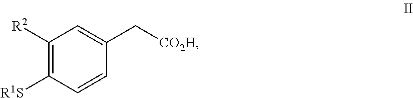

wherein R¹ and R² are as defined above, with performic acid to form a sulfone of the formula III,

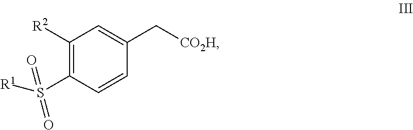

b) conversion of the sulfone of formula III with cyclopentane carbaldehyde and acetic anhydride in the presence of a base to form an acrylic acid derivative of formula IV,

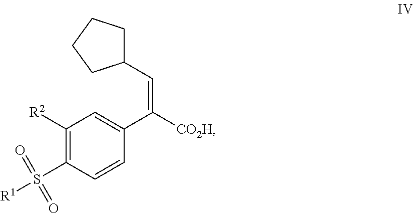

or a salt thereof, wherein R¹ and R² are as defined above; and;
c) the asymmetric hydrogenation of the acrylic acid derivative of the formula IV, or of a salt thereof, in the presence of a complex catalyst to form the propionic acid derivative of formula I.

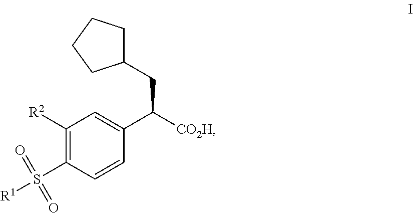

or of a salt thereof,
wherein R¹ and R² are as defined above.

The following definitions are set forth to illustrate and define the meaning and scope of the various terms used to describe the invention herein.

The term “C₁₋₆-alkyl”, alone or in combination with other groups, refers to a branched or straight-chain monovalent saturated aliphatic hydrocarbon radical of one to six carbon atoms, preferably one to four carbon atoms. This term is further exemplified by radicals as methyl, ethyl, n-propyl, isopropyl, n-butyl, s-butyl, t-butyl and pentyl or hexyl and its isomers.

The term “halogen-C₁₋₆-alkyl” refers to a halogen substituted C₁₋₆-alkyl radical wherein halogen has the meaning as outlined below. Preferred “halogen-C₁₋₆-alkyl” radicals are the fluorinated C₁₋₆-alkyl radicals such as CF₃, CH₂CF₃, CH(CF₃)₂, CH(CH₃)(CF₃), C₄F₉.

The term “C₃₋₈-cycloalkyl” group refers to a cycloalkyl group containing from 3 to 8 carbon atoms, such as cyclopropyl, cyclobutyl, cyclopentyl, cyclohexyl, cycloheptyl or cyclooctyl.

The term “C₁₋₆-alkoxy” refers to a branched or straight-chain monovalent saturated aliphatic hydrocarbon radical of one to six carbon atoms, preferably 1 to 4 carbon atoms attached to an oxygen atom. Examples of “alkoxy” are methoxy, ethoxy, propoxy, isopropoxy, butoxy, isobutoxy and hexyloxy. Preferred are the alkoxy groups specifically exemplified herein.

The alkyl chain of the alkoxy group can optionally be substituted, particularly mono-, di- or tri-substituted by alkoxy groups as defined above, preferably methoxy, or ethoxy or by aryl groups, preferably phenyl. Preferred substituted alkoxy group is the benzyloxy group.

The term “C₁₋₆-alkyl carbonyl” refers to C₁₋₆-alkyl substituted carbonyl group, preferably to a C₁₋₄-alkycarbonyl group. It includes for example acetyl, propanoyl, butanoyl or pivaloyl. Preferred alkyl carbonyl group is acetyl.

The term “C₁₋₆-alkyl carbonyl oxy” refers to a C₁₋₆-alkyl carbonyl substituted —O— group, preferably to a C₁₋₄-alkyl carbonyl substituted —O— group.

The term “mono- or di-C₁₋₆-alkyl-amino” refers to an amino group, which is mono- or disubstituted with C₁₋₆-alkyl, preferably C₁₋₄-alkyl. A mono-C₁₋₆-alkyl-amino group includes for example methylamino or ethylamino. The term “di-C₁₋₆-alkyl-amino” includes for example dimethylamino, diethylamino or ethylmethylamino. Preferred are the mono- or di-C₁₋₄-alkylamino groups specifically exemplified herein. It is hereby understood that the term “di-C₁₋₆-alkyl-amino” includes ring systems wherein the two alkyl groups together with the nitrogen atom to which they are attached form a 4 to 7 membered heterocycle which also may carry one further hetero atom selected from nitrogen, oxygen or sulfur.

The term “aryl”, alone or in combination with other groups, relates to a phenyl or naphthyl group, which can optionally be mono-, di-, tri- or multiply-substituted by halogen, hydroxy, CN, halogen-C₁₋₆-alkyl, NO₂, NH₂, N(H,C₁₋₆-alkyl), N(C₁₋₆-alkyl)₂, carboxy, aminocarbonyl, C₁₋₆-alkyl, alkoxy, C₁₋₆-alkyl carbonyl, C₁₋₆-alkylsulfonyl, SO₂-aryl, SO₃H, SO₃-alkyl, SO₂—NR′R″, aryl and/or aryloxy. Preferred aryl group usually is phenyl, however the preference for aryl may differ as indicated hereinafter for certain substituents.

The term “heteroaryl” relates to a heterocyclic aryl radical containing 1 to 3 heteroatoms in the ring with the remainder being carbon atoms. Suitable heteroatoms include, without limitation, oxygen, sulfur, and nitrogen. Exemplary heteroaryl groups include furanyl, thienyl, pyridyl, pyrrolyl, N-alkyl pyrrolo, pyrimidyl, pyrazinyl, imidazolyl, benzofuranyl, quinolinyl, and indolyl. Like the aryl group the heteroaryl group can optionally be mono-, di-, tri- or multiply-substituted by halogen, hydroxy, CN, halogen-C₁₋₆-alkyl, NO₂, NH₂, N(H,C₁₋₆-alkyl), N(C₁₋₆-alkyl)₂, carboxy, aminocarbonyl, C₁₋₆-alkyl, alkoxy, C₁₋₆-alkyl carbonyl, C₁₋₆-alkylsulfonyl, SO₂-aryl, SO₃H, SO₃-alkyl, SO₂—NR′R″, aryl and/or aryloxy.

The term “halogen” refers to a fluorine, chlorine, bromine or iodine atom, preferably to a chlorine atom.

### Step a)

The first step of the process of the present invention involves the oxidation of the sulfide of the formula II with performic acid to form a sulfone of the formula III.

The sulfides of the formula II are as a rule commercially available compounds.

The oxidation agent performic acid is as a rule produced in situ by adding hydrogen peroxide to formic acid at a temperature of 20° C. to 60° C. A higher temperature of 40° C. to 60° C. and slow dosing of hydrogen peroxide is preferred to avoid accumulation of performic acid in the reaction mixture (Ripin et al, Organic Process Research & Development 2007, 11, 762-765).

Usually formic acid is used as solvent for the sulfide of formula II, however an inert organic solvent, preferably a halogenated hydrocarbon such as methylene chloride may be used as co-solvent.

As a rule, after the reaction is completed and prior to the work up and the reaction mixture is quenched with a reducing agent such as sodium bisulfite.

The isolation of the sulfone of formula III can happen according to methods known to the skilled in the art, usually by removing the excess formic acid and crystallizing the sulfone from water.

In a preferred embodiment the sulfide of formula II with R¹=methyl and R²=chlorine is used.

### Step b)

Step b) involves the conversion of the sulfone of formula III with cyclopentane carbaldehyde and acetic anhydride in the presence of a base to an acrylic acid derivative of the formula IV.

The cyclopentane carbaldehyde can be applied in an amount of 1.0 to 5.0 equivalents related to the sulfone of formula III, preferably in an amount of 1.5 equivalents

The acetic anhydride can be applied in an amount of 1.0 to 5.0 equivalents related to the sulfone of formula III, preferably 2.5 equivalents.

The base is usually an alkali acetate, preferably sodium acetate or potassium acetate.

The reaction can be run without additional organic solvent; however a suitable organic solvent such as tetrahydrofurane, acetonitrile, ethyl acetate or acetone, preferably tetrahydrofurane or acetone may be added.

The conversion is usually performed at a reaction temperature of from 20° C. to 100° C., preferably from 30° C. to 60° C.

The acrylic acid derivative of formula IV will preferably be isolated in the form of the dicyclohexylamine salt or in the form of an alkali metal salt such as the Li-, Na-, K-salt. Preferred alkali metal salt is the Na-salt.

The dicyclohexylamine salt can be obtained by converting the free acid of the acrylic acid derivative of formula IV with the dicyclohexyl amine in the presence of a suitable organic solvent such as acetone at ambient temperature.

The free acid, on the other hand, can be obtained by acidifying e.g. the dicyclohexylamine salt with an aqueous mineral acid.

The alkali metal salt can either be obtained from the dicyclohexylamine salt e.g. by liberating the free acid with an aqueous mineral acid and subsequent conversion with the suitable alkali alkoxide or by direct conversion of the free acid with the alkali alkoxide.

In a preferred embodiment the substituents of the double bond in the acrylic acid derivative of the formula IV have an (E)-configuration.

In a preferred embodiment the sulfone of formula III with R¹=methyl and R²=chlorine is used.

### Step c)

Step c) involves the asymmetric hydrogenation of the acrylic acid derivative of the formula IV, or of a salt thereof, in the presence of a complex catalyst to form the propionic acid derivative of formula I or of a salt thereof.

In an embodiment, the acrylic acid derivative of the formula IV used for the asymmetric hydrogenation is selected from the free acid, the dicyclohexylamine salt or from an alkali metal salt thereof.

The complex catalyst can be selected from compounds of formulas

Ru(Z)₂D (VIa)

[Ru(Z)₂₋ₚ(D)(L)ₘ](Y)ₚ (VIb)

[Ru(D)(L)₂](Y)₂ (VIc)

[M(D)LX] (VId)

[M(D)L]⁺Y⁻ (VIe)

wherein
each Z is selected from the group consisting of hydrogen, halogen, η⁵-2,4-pentadienyl, η⁵-2,4-dimethyl-pentadienyl and the group A-COO⁻ wherein
A is selected from the group consisting of C₁₋₆-alkyl, aryl, halogenated C₁₋₆-alkyl and halogenated aryl;
Y is a non-coordinating anion;
D is a chiral phosphine ligand;
L is a neutral ligand;

### M is Iridium or Rhodium

X is a halogen atom;
m is an integer from 1 to 3;
p is 1 or 2.

In a preferred embodiment the phosphine ligand D is selected from the group consisting of:

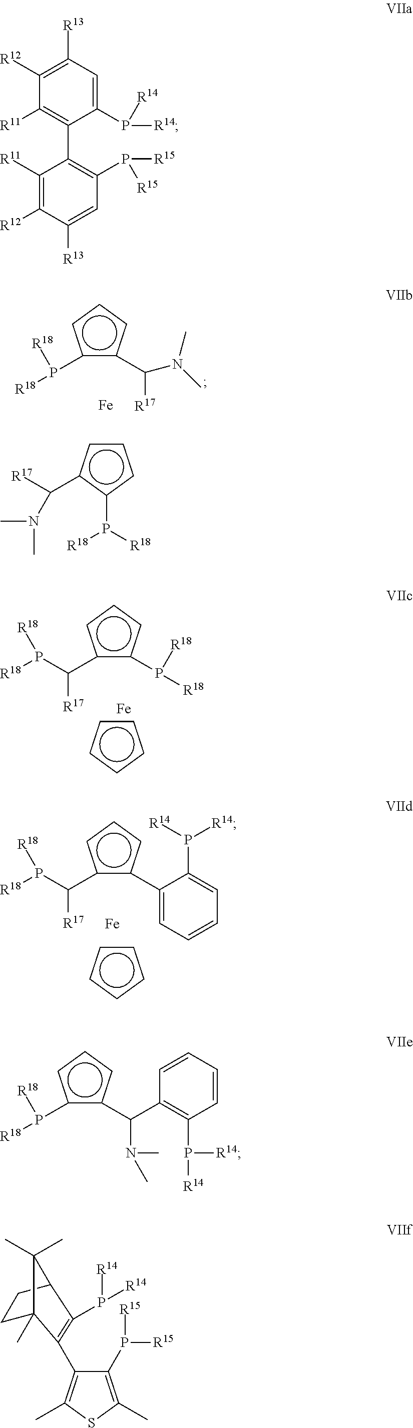

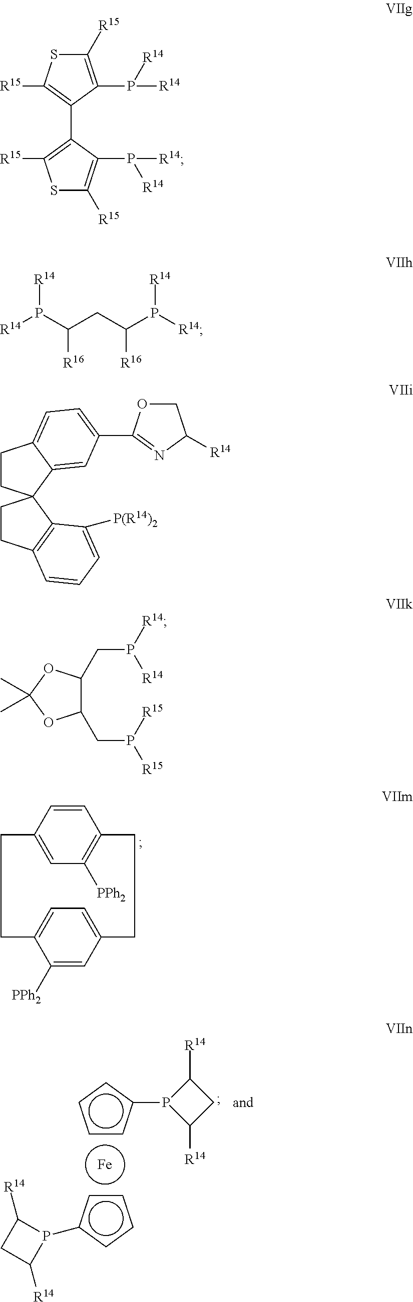

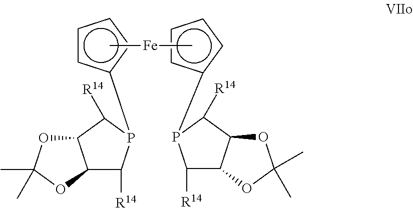

wherein
R¹¹ is selected from the group consisting of C₁₋₆-alkyl, C₁₋₆-alkoxy, hydroxy and C₁₋₆-alkyl carbonyl oxy;
R¹² and R¹³ independently of each other are selected from the group consisting of hydrogen, C₁₋₆-alkyl, C₁₋₆-alkoxy and di-(C₁₋₆-alkyl)amino; or
R¹¹ and R¹² which are attached to the same phenyl group, or
R¹² and R¹³ which are attached to the same phenyl group, taken together, are —X—(CH₂)ᵣ—Y—, wherein X is —O— or —C(O)O—, Y is —O— or —N(C₁₋₆-alkyl)- and r is an integer from 1 to 6, or a CF₂ group,
or both R¹¹s, taken together, are —O—(CH₂)ᵣ—O— or O—CH(CH₃)—(CH₂)ᵣ—CH(CH₃)—O—, wherein r is an integer from 1 to 6, or
R¹¹ and R¹², or R¹² and R¹³, together with the carbon atoms to which they are attached, form a naphthyl, tetrahydronaphthyl or dibenzofuran ring;
R¹⁴ and R¹⁵ independently of each other are selected from the group consisting of C₁₋₆-alkyl, C₃₋₈-cycloalkyl, phenyl, naphthyl and heteroaryl, substituted with 0 to 7 substituents independently selected from the group consisting of C₁₋₆-alkyl, C₁₋₆-alkoxy, di(C₁₋₆-alkyl)amino, morpholino, phenyl and tri(C₁₋₆-alkyl)silyl, carboxy, and C₁₋₆-alkoxycarbonyl;
R¹⁶ is C₁₋₆-alkyl;
R¹⁷ is C₁₋₆-alkyl; and
R¹⁸ is selected from the group consisting of aryl, heteroaryl, C₃₋₈-cycloalkyl and C₁₋₆-alkyl.

In a more preferred embodiment the phosphine ligand D is selected from the group consisting of compounds of formula VIIa, VIIc, VIIh, VIIi and VIIo.

Y is preferably selected from the group consisting of halides, AsF₆⁻, BF₄⁻, ClO₄⁻, SbF₆⁻, PF₆⁻, B(phenyl)₄⁻, B(3,5-di-trifluoromethyl-phenyl)₄⁻, CF₃SO₃⁻, and C₆H₅SO₃⁻.

Y more preferably is selected from the group consisting of BF₄⁻, B(3,5-di-trifluoromethyl-phenyl)₄⁻ and Cl⁻.

L is preferably selected from the group consisting of ethylene, propylene, cyclooctene, 1,3-hexadiene, 1,5-hexadiene, bicyclo-[2.2.1]hepta-2,5-diene, (Z,Z)-1,5-cyclooctadiene, benzene, hexamethylbenzene, 1,3,5-trimethylbenzene, p-cymene and solvents selected from the group consisting of tetrahydrofuran, N,N-dimethylformamide, acetonitrile, dimethylsulfoxide, benzonitrile, acetone, methanol and pyridine.

L more preferably stands for (Z,Z)-1,5-cyclooctadiene or acetonitrile.

X is a halide such as Cl⁻, Br⁻ or I⁻, preferably Cl⁻.

A preferably is methyl or trifluoromethyl.

Z preferably is η⁵-2,4-dimethyl-pentadienyl, iodide or acetyl

m preferably is 1

p preferably is 1.

In a more preferred embodiment the complex catalyst is selected from:

a compound of formula VIa in which both Zs are acetate and D is a compound of formula VIIa (catalyst type VIa/Ru-1);
a compound of formula VIa in which both Zs are trifluoroacetate and D is a compound of formula VIIa (catalyst type VIa/Ru-2);
a compound of formula VIa in which both Zs are Cl and D is a compound of formula VIIa (catalyst type VIa/Ru-3);
a compound of formula VIa in which both Zs are acetate and D is a compound of formula VIIc (catalyst type VIa/Ru-4);
a compound of formula VIa in which both Zs are acetate and D is a compound of formula VIIg (catalyst type VIa/Ru-5);
a compound of formula VIa in which both Zs are acetate and D is a compound of formula VIIn (catalyst type VIa/Ru-6);
a compound of formula VIa in which both Zs are acetate and D is a compound of formula VIIo (catalyst type VIa/Ru-7);
a compound of formula VIb in which Z is halogen, D is a compound of formula VIIa, L is C₆H₆, Y is halogen, p is 1 and m is 1 (catalyst type VIb/Ru-8);
a compound of formula VIa in which one Z is η⁵-2,4-dimethyl-pentadienyl and the other is I and D is a compound of formula VIIe (catalyst type VIa/Ru-9);
a compound of formula VIb in which Z is H, D is a compound of formula VIIa, L is C₆H₆, Y is BF₄⁻, p is 1 and m is 1 (catalyst type VIb/Ru-10);
a compound of formula VIb in which Z is halogen, D is a compound of formula VIIa, L is p-cymene, Y is halogen, p is 1 and m is 1 (catalyst type VIb/Ru-11);
a compound of formula VIb in which Z is η⁵-2,4-dimethyl-pentadienyl, D is a compound of formula VIIc, L is CH₃CN, Y is BF₄⁻, p is 1 and m is 1 (catalyst type VIb/Ru-12);
a compound of formula VIc in which D is a compound of formula VIIa, one L is p-cymene and the other L is CH₃CN, and both Ys are BF₄⁻ (catalyst type VIc/Ru-13);
a compound of formula VIb in which both Zs are Cl, D is a compound of formula VIIa, L is N, N-dimethylformamide, p is 0, and m is 1 (catalyst type VIb/Ru-14);
a compound of formula VId wherein M is Ir, D is a compound of formula VIIh, L is 1,5-cyclooctadiene, and X is Cl (catalyst type VId/Ir-1);
a compound of formula VId wherein M is Rh, D is a compound of formula VIIb, L is 1,5-cyclooctadiene, and X is Cl (catalyst type VId/Rh-1);
a compound of formula VId wherein M is Rh, D is a compound of formula VIIb, L is 1,5-cyclooctadiene, and X is trifluoroacetate (catalyst type VId/Rh-2);
a compound of formula VId wherein M is Rh, D is a compound of formula VIIb, L is 1,5-cyclooctadiene, and X is acetylacetonate (catalyst type VId/Rh-3);
a compound of formula VIe wherein M is Ir, D is a compound of formula VIIa, L is 1,5-cyclooctadiene, and Y is BF₄⁻ (catalyst type VIe/Ir-2),
a compound of formula VIe wherein M is Ir, D is a compound of formula VIIb, L is 1,5-cyclooctadiene, and Y is BF₄⁻ (catalyst type VIe/Ir-3);
a compound of formula VIe wherein M is Ir, D is a compound of formula VIIb, L is 1,5-cyclooctadiene, and Y is tetrakis[3,5-bis(trifluoromethyl)phenyl]borate (catalyst type VIe/Ir-4);
a compound of formula VIe wherein M is Ir, D is a compound of formula VIIh, L is 1,5-cyclooctadiene, and Y is BF₄⁻ (catalyst type VIe/Ir-5);
a compound of formula VIe wherein M is Ir, D is a compound of formula VIIh, L is 1,5-cyclooctadiene, and Y is tetrakis[3,5-bis(trifluoromethyl)phenyl]borate (catalyst type VIe/Ir-6);
a compound of formula VIe wherein M is Ir, D is a compound of formula VIIi, L is 1,5-cyclooctadiene, and Y is tetrakis[3,5-bis(trifluoromethyl)phenyl]borate (catalyst type VIe/Ir-7);
a compound of formula VIe wherein M is Rh, D is a compound of formula VIIa, L is 1,5-cyclooctadiene, and Y is BF₄⁻ (catalyst type VIe/Rh-4);
a compound of formula VIe wherein M is Rh, D is a compound of formula VIIb, L is 1,5-cyclooctadiene, and Y is BF₄⁻ (catalyst type VIe/Rh-5);
a compound of formula VIe wherein M is Rh, D is a compound of formula VIIb, L is 1,5-cyclooctadiene, and Y is SbF₆ (catalyst type VIe/Rh-6);
a compound of formula VIe wherein M is Rh, D is a compound of formula VIIb, L is 1,5-cyclooctadiene, and Y is CF₃SO₃⁻ (catalyst type VIe/Rh-7); and
a compound of formula VIe wherein M is Rh, D is a compound of formula VIIh, L is 1,5-cyclooctadiene, and Y is BF₄⁻ (catalyst type VIe/Rh-8).

In an even more preferred embodiment the complex catalyst is selected from the catalyst types VIa/Ru-1, VIa/Ru-4, VIa/Ru-7, VIb/Ru-8, VId/Ir-1, VIe/Ir-5, VIe/Ir-7, VIe/Rh-6 or VIe/Rh-8.

Preferred catalysts for the asymmetric hydrogenation of the free acid of the acrylic acid derivative of the formula IV are:
[1-[(1S)-1-[Bis(1,1-dimethylethyl)phosphino]ethyl]-(R)-2-(diphenyl phosphino)ferrocene)-(η⁵-2,4-dimethylpentadienyl)-(N-acetonitrile)]ruthenium(II)]tetrafluoroborate; ([Ru((S,R)-tBu-Josiphos)(2,4-Me-C₅H₅)(CH₃CN)]BF₄; and
[(S)-(6,6′-Dimethoxybiphenyl-2,2′-diyl)bis[bis(3,5-dimethylphenyl)-phosphineo]bisacetato]ruthenium(II); ([Ru(OAc)₂((S)-3,5-Xyl-MeOBIPHEP)]).

Preferred catalysts for the asymmetric hydrogenation of the dicyclohexylamine salt of the acrylic acid derivative of the formula IV are:
[1-[(1S)-1-[Bis(1,1-dimethylethyl)phosphino]ethyl]-(R)-2-(diphenyl phosphino)ferrocene)-(η⁵-2,4-dimethylpentadienyl)-(N-acetonitrile)]ruthenium(II)]tetrafluoroborate; ([Ru((S,R)-tBu-Josiphos)(2,4-Me-C₅H₅)(CH₃CN)]BF₄; and
1-[(1S)-1-[Bis(1,1-dimethylethyl)phosphino]ethyl]-(R)-2-(diphenyl phosphino)ferrocene)(bisacetato)ruthenium(II); ([Ru(OAc)₂(S,R)-tBu-Josiphos)]).
[((1S,3S)-1,3-Dimethyl-1,3-propanediyl)bis(bis(3,5-dimethylphenyl)phosphino)(1,5-cyclooctadiene)Iridium(I)]tetrafluoroborate; ([Ir(COD)((S,S)-3,5-Xyl-Skewphos)]BF₄;
[(S)-(6,6′-Dimethoxybiphenyl-2,2′-diyl)bis[bis(3,5-dimethylphenyl)-phosphineo]bisacetato]ruthenium(II); ([Ru(OAc)₂((S)-3,5-Xyl-MeOBIPHEP)]).

Preferred catalysts for the asymmetric hydrogenation of the sodium salt of the acrylic acid derivative of the formula IV are:
[((1S,3S)-1,3-Dimethyl-1,3-propanediyl)bis(bis(3,5-dimethylphenyl)phosphino)(1,5-cyclooctadiene)Iridium(I)]tetrafluoroborate; (([Ir(COD)((S,S)-3,5-Xyl-Skewphos)]BF₄);
[1-[(1S)-1-[Bis(1,1-dimethylethyl)phosphino]ethyl]-(R)-2-(diphenyl phosphino)ferrocene)-(η⁵-2,4-dimethylpentadienyl)-(N-acetonitrile)]ruthenium(II)]tetrafluoroborate; ([Ru((S,R)-tBu-Josiphos)(2,4-Me-C₅H₅)(CH₃CN)]BF₄; and
[(S)-(6,6′-Dimethoxybiphenyl-2,2′-diyl)bis[bis(3,5-dimethylphenyl)-phosphineo]bisacetato]ruthenium(II); ([Ru(OAc)₂((S)-3,5-Xyl-MeOBIPHEP)]);
1,1′-Bis-[(2S,3S,4S,5S)-3,4-O-isopropylidene-3,4-dihydroxy-2,5-dimethylphosphanyl]ferrocene]bisacetato]ruthenium(II); ([Ru(OAc)₂((S,S,S,S)-Me-f-KetalPhos)]).

In the ruthenium complex catalysts of the formula VIa, VIb and VIc the ruthenium is characterized by the oxidation number II. These complexes can in principle be prepared in a manner known per se. They can be isolated or used directly (in situ preparation) e.g. according to B. Heiser et al., Tetrahedron: Asymmetry 1991, 2, 51 or N. Feiken et al., Organometallics 1997, 16, 537 or J.-P. Genet, Acc. Chem. Res. 2003, 36, 908 or K. Mashima et al., J. Org. Chem. 1994, 53, 3064, M. P. Fleming et al., U.S. Pat. No. 6,545,165 B1, and references cited therein as well as O. Briel et al. in Catalysis of Organic Reactions, CRC Press, Boca Raton, 2009 specifically for the ferrocene-based Ru-complexes.

In the rhodium and iridium complex catalysts of the formula VId and VIe the metal is characterized by the oxidation number I. They can be prepared, for example, by reaction of metal precursors such as e.g. di-η⁴-chloro-bis[η⁴-4-(Z,Z)-1,5-cyclo-octadiene]dirhodium(I) ([Rh(cod)Cl]₂), di-μ-chloro-bis[η⁴-norbornadiene]-dirhodium(I) ([Rh(nbd)Cl]₂), bis[η⁴-(Z,Z)-1,5-cyclooctadiene]rhodium tetra-fluoroborate ([Rh(cod)₂]BF₄) or bis[η⁴-(Z,Z)-cyclooctadiene]rhodium perchlorate ([Rh(cod)₂]ClO₄) or the corresponding Iridium analogues with a chiral phosphine ligand in a suitable inert organic or aqueous solvent (e.g. according to the methods described in J. Am. Chem. Soc. 1971, 93, 2397 or E. Jacobsen, A. Pfaltz, H. Yamamoto (Eds.), Comprehensive Asymmetric Catalysis I-III, Springer Verlag Berlin (1999) and references cited therein).

Rhodium, iridium or ruthenium complex catalysts as described above can also be prepared in situ, i.e. just before use and without isolation. The solution in which such a catalyst is prepared can already contain the substrate for the enantioselective hydrogenation or the solution can be mixed with the substrate just before the hydrogenation reaction is initiated.

In general the asymmetric hydrogenation is performed in an organic solvent at a reaction temperature between 10° C. and 100° C., preferably from 20° C. to 60° C. and a pressure between 1 and 180 bar, preferably between 20 bar and 70 bar.

The substrate/catalyst ratio (S/C) is commonly between 5 and 100,000, preferentially between 1000-75,000.

For Ru-type catalysts the S/C ratio as a rule ranges from 20 to 75,000 and for Ir- and Rh-type catalysts from 20 to 2500.

Suitable solvents for the hydrogenation with ruthenium complexes are alcohols, hydrocarbons, chlorinated hydrocarbons, fluorinated and polyfluorinated aliphatic or aromatic hydrocarbons, supercritical or liquid carbon dioxide, THF, water or mixtures thereof. Additives such as e.g. polyethylene glycol (PEG) may be added. Preferred solvents are alcohols, preferably methanol, chlorinated hydrocarbons, preferably methylene chloride and THF.

Suitable solvents for iridium and rhodium complexes are alcohols; aromatic hydrocarbons, such as benzene, toluene, trifluoro toluene; halogenated hydrocarbons, such as dichloromethane, dichlororethane, etc.; polyalcohols such as ethylene glycol; amides such as DMF, DMA, and N-methylpyrrolidinone; supercritical or liquid carbon dioxide; acetonitrile; water; or DMSO. Preferred solvents are alcohols, such as methanol or chlorinated hydrocarbons such as methylene chloride.

The solvents can be used alone or as mixture of solvents mentioned above.

The asymmetric hydrogenation is usually performed in a basic environment.

No additional base is necessary when the asymmetric hydrogenation is carried out with the alkali metal salt or the dicyclohexylamine salt as substrate.

It was found that no additional base is deemed necessary when the free acid of formula IV is converted in the presence of a ruthenium complex catalyst.

The acrylic acid derivative of formula IV can be employed as an acid in the asymmetric hydrogenation. It can however be convenient to convert it, at least in part, to its salt by addition of a base. Preferably the salt is pre-formed, isolated and purified before the hydrogenation. However, it can be prepared in situ shortly before the hydrogenation.

Suitable bases can be selected from tertiary amines, such as NEt₃ and i-Pr₂Net; secondary amines, such as iPr₂NH or Cy₂NH; primary amines, such as C₆H₅CH₂NH₂, 1-phenyl-benzylamine, and ((R) or (S)), cyclohexyl-ethylamine ((R) or (S)); diamines, such as ethylene diamine and tetramethylethylene diamine; inorganic bases such as NaOH and KOH; salts of carboxylic acids, such as NaOAc; salts of alcoholates, such as NaOEt, and tetrasubstituted ammonium salts, such as Bu₄NX (X=F, Cl, Br, I).

Preferred bases are NEt₃, Cy₂NH, (R)-Cyclohexyl-ethyl-amine, and NaOH. More preferred bases are NaOH and Cy₂NH.

The amount of base applied is in the range of from 0.1 to 100 equivalents, preferably from 0.15 to 1.0 equivalents.

In a preferred embodiment, the dicyclohexylamine salt or the sodium salt of the acrylic acid derivative of formula IV with R¹=methyl and R²=chlorine is used.

### EXAMPLES

### Abbreviations

NCMe/CH₃CN=acetonitrile
TFA=trifluoro acetate

### Otf=CF₃SO₃⁻

OAc=acetate
acac=acetylacetonate
p-Cym=p-cymene
Xyl=3,5-dimethylphenyl

### Cyp=Cyclopentyl

### Cy=Cyclohexyl

COD=1,5-cyclooctadiene
BARF=tetrakis[3,5-bis(trifluoromethyl)phenyl]borate
2,4-C₅H₅=η⁵-2,4-dimethylpentadienyl
THF=tetrahydrofuran

### DMF=N,N-dimethylformamide

### Tol=Toluene

1,2-DME=1,2-dimethoxy ethane

### TMS=Trimethylsilyl

S/C=substrate-to-catalyst molar ratio
PEG=Polyethylene glycol

### A. Sulfide Oxidation

### Example A1

### (3-Chloro-4-methanesulfonyl-phenyl)acetic acid

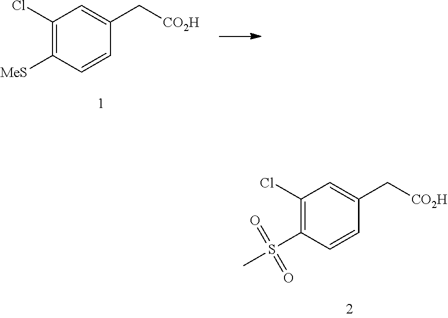

In a 750-ml four-necked flask equipped with a mechanical stirrer, a Pt-100 thermometer, a rubber septum, and a glass stopper, 100 g sulfide 1 (461 mmol, 1.0 equiv) are suspended in formic acid (500 ml) and the mixture is warmed to 50° C. 100 ml hydrogen peroxide (30% aqueous solution, 978 mmol, 2.1 equiv) are added over 6 hours via syringe pump. After the end of the addition, stirring at 50° C. is maintained for 16 hours.

The reaction mixture is cooled to ambient temperature and quenched with 18.6 ml sodium bisulfite (39% aqueous solution, 92 mmol, 0.2 equiv) under ice cooling. The mixture is stirred at ambient temperature for 20 minutes and 430 ml formic acid are stripped under reduced pressure at 50° C. To the resulting solution is added 500 ml water over 40 minutes, whereupon crystallization occurs. The white suspension is stirred at ambient temperature for 20 hours and at 0° C. for 3 hours. The crystals are filtered off, washed with water (100 ml) and dried under reduced pressure (10 mbar) at 50° C. for 24 hours. 106 g of 2 (92% yield) are obtained as white crystals (m.p. 125° C.).

¹H-NMR (CDCl₃): δ 8.11 (d, 1H), 7.52 (d, 1H), 7.40 (dd, 1H), 3.74 (s, 2H), 3.27 (s, 3H)

### B. Sulfone Conversion/Salt Formation

### Example B1

### (E)-2-(3-Chloro-4-methanesulfonyl-phenyl)-3-cyclopentyl-acrylic acid Dicyclohexylamine Salt

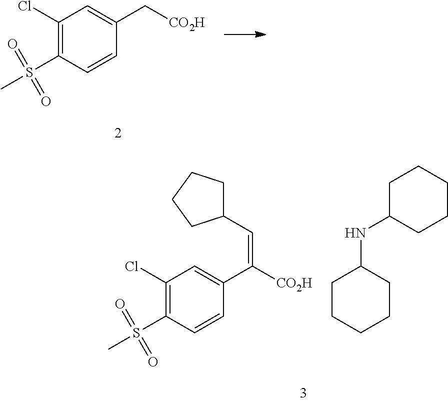

In a 200-ml four-necked flask equipped with a mechanical stirrer, a reflux condenser (with inert gas in/outlet), a Pt-100 thermometer, and a 250-ml addition funnel, 10 g sulfone 2 (40.2 mmol, 1.0 equiv) are dissolved in tetrahydrofuran (20 ml) and 9.5 ml acetic anhydride (101 mmol, 2.5 equiv), 6.6 ml cyclopentane carboxaldehyde (60.3 mmol, 1.5 equiv), and 3.32 g sodium acetate (40.2 mmol, 1.0 equiv) are added. The mixture is stirred at 40° C. for 25 h, whereupon water (60 ml) and 4-dimethylaminopyridine (50 mg, 0.01 equiv) is added at 40° C. The resulting mixture is stirred at 40° C. for 2 hours. The pH is adjusted to pH=6 using 10M aqueous NaOH (18 ml) under ice cooling. The organics are evaporated under reduced pressure at 40° C. and the remaining aqueous layer is extracted twice with each 50 ml TBME. The combined organic phases are washed three times each time with 30 ml H₂O, dried over anhydrous Na₂SO₄, and concentrated under reduced pressure. The residue is azeotropically dried with toluene (30 ml), giving 14.5 g of the crude product.

In a 150-ml four-necked equipped with a mechanical stirrer, an addition funnel, an inert gas in/outlet, and a glass stopper, the crude product is dissolved in acetone (60 ml) and 7.20 ml dicyclohexylamine (36.2 mmol, 0.9 equiv) are added over 3 minutes, whereupon crystallization occurs. The off-white suspension is stirred for 17 hours at ambient temperature followed by 5 hours at 0° C. The crystals are filtered off, washed with 20 ml acetone/heptane 1:1 and dried under reduced pressure (10 mbar) at 50° C. for 19 hours. 15.0 g of (E)-3 (73% yield) are obtained as white crystals (m.p. 193-196° C.).

¹H-NMR (CDCl₃): δ 8.09 (d, 1H), 7.42 (d, 1H), 7.27 (dd, 1H), 6.78 (d, 1H), 3.28 (s, 3H), 2.81 (m, 2H), 2.32 (m, 1H), 1.92 (m, 4H), 1.8-1.05 (m, 24H)

### Example B2

### (E)-2-(3-Chloro-4-methanesulfonyl-phenyl)-3-cyclopentyl-acrylic acid Sodium Salt

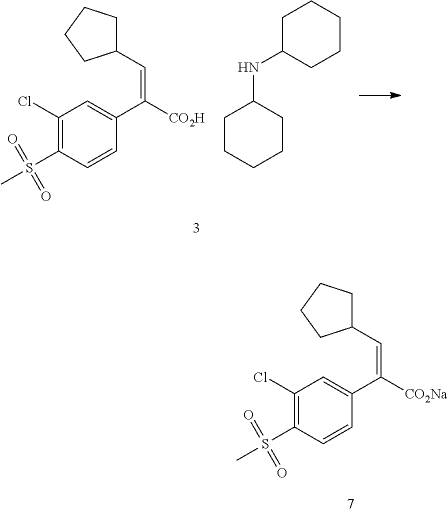

In a 3-1 separation funnel, 50 g salt (E)-3 (98 mmol, 1 equiv) are treated with dichloromethane (500 ml) and 0.1 M aqueous hydrochloric acid (11). The layers are separated and the organic layer is washed twice with 0.2 M aqueous hydrochloric acid (2×0.5 l). The organic phase is dried over anhydrous Na₂SO₄, filtered, and concentrated under reduced pressure to give 32.13 g of a white foam.

The foam is dissolved in 2-PrOH (150 ml) and sodium methoxide (5.4 M in MeOH, 18.1 ml, 97.7 mmol, 1 equiv) is added over 20 minutes. The mixture is warmed to 50° C. to give a clear mixture and subsequently cooled to ambient temperature. After stirring at ambient temperature for 60 h and at 0° C. for 6 h, the crystals are filtered off, washed with cold 2-PrOH (20 ml) and dried under reduced pressure (10 mbar) at 60° C. for 24 h. 30.31 g of (E)-7 (88% yield; 84% yield corrected for residual 2-PrOH) are isolated as white crystals (m.p. 214° C.).

¹H-NMR (DMSO): δ 7.92 (d, 1H), 7.41 (d, 1H), 7.27 (dd, 1H), 6.48 (d, 1H), 3.36 (s, 3H), 2.25 (m, 1H), 1.7-1.25 (m, 8H)

### Example B3

### (E)-2-(3-Chloro-4-methanesulfonyl-phenyl)-3-cyclopentyl-acrylic acid Sodium Salt

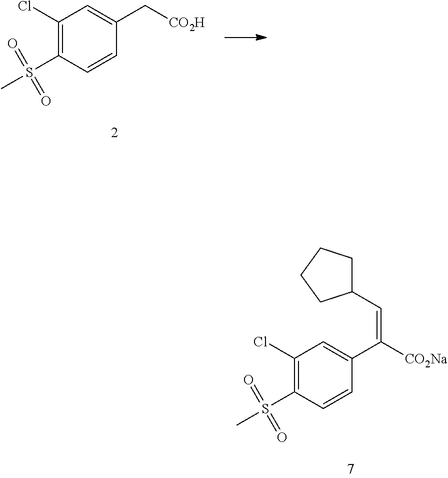

In a 200-ml four-necked flask equipped with a mechanical stirrer, a reflux condenser (with inert gas in/outlet), a Pt-100 thermometer, and a 250-ml addition funnel, 10 g sulfone 2 (40.2 mmol, 1.0 equiv) are dissolved in tetrahydrofurane (20 ml) and 9.5 ml acetic anhydride (101 mmol, 2.5 equiv), 6.6 ml cyclopentane carboxaldehyde (60.3 mmol, 1.5 equiv), and 3.32 g sodium acetate (40.2 mmol, 1.0 equiv) are added. The mixture is stirred at 40° C. for 24 h, whereupon water (60 ml) and 4-dimethylaminopyridine (50 mg, 0.01 equiv) are added at 40° C. The resulting mixture is stirred at 40° C. for 2 hours. The pH was adjusted to pH=6 using 2 M aqueous NaOH (30 ml) and 5 M aqueous NaOH (25 ml) under ice cooling. The organics are evaporated under reduced pressure at 40° C. and the remaining aqueous layer is extracted twice with each 50 ml TBME. The combined organic phases are washed three times with each 20 ml H₂O, dried over anhydrous Na₂SO₄, and concentrated under reduced pressure. The residue is azeotropically dried with toluene (30 ml), giving 14.7 g of the crude product.

The crude product is dissolved in 2-PrOH (66 ml) and sodium methoxide (5.4 M in MeOH, 6.70 ml, 36.2 mmol, 0.9 equiv) is added over 20 minutes. The mixture is warmed to 50° C. to give a clear mixture and subsequently cooled to ambient temperature. After stirring at ambient temperature for 16 h, the crystals are filtered off, washed with cold 2-PrOH (10 ml) and dried under reduced pressure (10 mbar) at 60° C. for 18 h. 6.84 g of (E)-7 (49% yield) are isolated as off-white crystals.

### Example B 4

### (E)-2-(3-Chloro-4-methanesulfonyl-phenyl)-3-cyclopentyl-acrylic acid

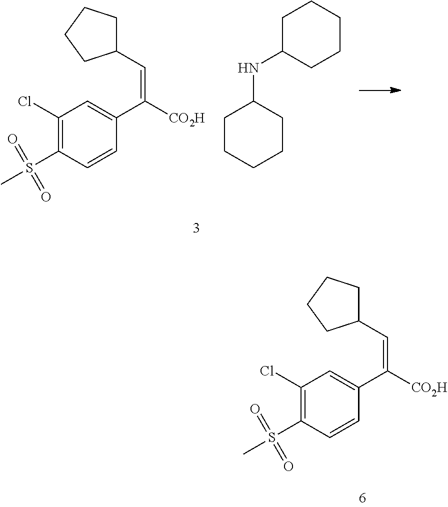

In a 3-1 separation funnel, 50 g salt (E)-3 (98 mmol, 1 equiv) are treated with dichloromethane (500 ml) and 0.1 M aqueous hydrochloric acid (11). The layers are separated and the organic layer is washed twice with 0.2 M aqueous hydrochloric acid (2×0.5 l). The organic phase is dried over anhydrous Na₂SO₄, filtered, and concentrated under reduced pressure to give 31.52 g of a white foam.

The solid is suspended in toluene (95 ml) and dissolved by heating to 55° C. Crystallization occurs upon cooling to ambient temperature and seeding. Stirring is maintained for 66 h, followed by 7 h at 0° C. The crystals are filtered off, washed with cold toluene (25 ml) and dried under reduced pressure (10 mbar) at 60° C. 26.57 g of

(E)-6 (84% yield) are isolated as white crystals (m.p. 76-78° C.).

¹H-NMR (CDCl₃): δ 8.15 (d, 1H), 7.41 (d, 1H), 7.30 (dd, 1H), 7.20 (d, 1H), 3.30 (s, 3H), 2.40 (m, 1H), 1.7-1.4 (m, 8H)

### C. Asymmetric Hydrogenation

Ferrocenyl phosphine ligands of the Josiphos, Mandyphos and Taniaphos families are commercially available from Solvias AG, CH-4002 Basel. The corresponding ruthenium complexes are commercially available from Umicore AG, D-63457 Hanau-Wolfgang or can be prepared according to O. Briel et al. in “Catalysis of Organic Reactions”, 2009, 209, CRC Press, Boca Raton. Skewphos and Xyl-Skewphos are commercially available from Digital Specialty Chemicals, 470 Coronation Drive, Toronto, Ontario, Canada M1E 4Y4. All BIPHEP and MeOBIPHEP types of ligands are either commercially available from Solvias AG, CH-4002 Basel or can be prepared according to the examples or methods as described in patent application documents EP 0 398 132, WO 92/16535, EP 0 104 375 or EP 0 580 331. Segphos and Xyl-Segphos derivatives as well as the Ru-Segphos-complexes are commercially available from Sigma-Aldrich-Fluka AG, CH-9471 Buchs. TMBTP is commercially available from Chemi S.p.A., Via dei Lavoratori, Cinasello Balsamo, Milano 20092, Italy, or can be prepared according to P. Antognazza, T. Benincori, E. Brenna, E. Cesarotti, F. Sannicolo, EP 95/02647 CAN 124:317487 patent to Italfarmaco Sud S.p.A. Italy (7.7.1995). Me-f-KetalPhos is commercially available from Chiral Quest, Princeton Corporate Plaza, Monmouth Jct., NJ08852, USA. The oxazoline-monophosphine ligands (SIPHOX ligands) and their corresponding iridium complexes are commercially available from Nankai University, Tianjin 300071 China or can be prepared according to Q. L. Zhou et al. J. Am. Chem. Soc. 2008, 130, 8584.

The following list provides the chemical names for the acronyms of the chiral phosphine ligands used:

| Acronyms | Chemical Name |
|---|---|
| MeOBIPHEP | (6,6′-Dimethoxybiphenyl-2,2′-diyl)bis(diphenyl- |
| | phosphine) |
| 3,5-Xyl-MeOBIPHEP | (6,6′-Dimethoxybiphenyl-2,2′-diyl)bis[bis(3,5- |
| | dimethylphenyl)phosphine] |
| DiMeOBIPHEP | 6,6′-Bis-diphenylphosphino-2,3,2′,3′-tetramethoxy- |
| | biphenyl |
| 3,5-tBu-4-MeO-MeOBIPHEP | (6,6′-Dimethoxybiphenyl-2,2′-diyl)bis[bis(3,5-tert- |
| | butyl-4-methoxy-phenyl)phosphine] |
| 3,5-tBu-MeOBIPHEP | (6,6′-Dimethoxybiphenyl-2,2′-diyl)bis[bis(3,5-tert- |
| | butyl-phenyl)phosphine] |
| BIPHEMP | (6,6′-Dimethylbiphenyl-2,2′-diyl)bis(diphenyl- |
| | phosphine) |
| TriMeOBIPHEP | 6,6′-Bis-diphenylphosphino-2,3,4,2′,3′,4′- |
| | hexamethoxy-biphenyl |
| pTol-BIPHEMP | (6,6′-Dimethylbiphenyl-2,2′-diyl)bis(di(4- |
| | methylphenyl)phosphine) |
| 3,5-iPr-MeOBIPHEP | (6,6′-Dimethoxybiphenyl-2,2′-diyl)bis[bis(3,5-di-iso- |
| | propyl)-phenyl)phosphine] |
| SKEWPHOS | 2,4-Bis(diphenylphosphino)pentane |
| 3,5-Xyl-Skewphos | 2,4-Bis(di(3,5-dimethyl)phenylphosphino)pentane |
| 3,4-Xyl-MeOBIPHEP | (6,6′-Dimethoxybiphenyl-2,2′-diyl)bis[bis(3,4- |
| | dimethyl-phenyl)phosphine] |
| 6-MeO-2-Naphthyl-BIPHEMP | (6,6′-Dimethylbiphenyl-2,2′-diyl)bis(di-2-(6- |
| | methoxy)naphthylphosphine) |
| SEGPHOS | 5,5′-Bis(diphenylphosphino)-4,4′-bi-1,3-benzodioxole |
| TMBTP | 2,2′,5,5′-Tetramethyl-4,4′-bis(diphenylphosphino)- |
| | 3,3′-bithiophene |
| (6-MeO-2-Naphthyl)-MeOBIPHEP | (6,6′-Dimethoxybiphenyl-2,2′-diyl)bis(di-2-(6- |
| | methoxy)naphthylphosphine) |
| (2-Thienyl)-MeOBIPHEP | (6,6′-Dimethoxybiphenyl-2,2′-diyl)bis[bis(2-thienyl)- |
| | phosphine] |
| Taniaphos (PPPhCHNMe₂-F-PP) | 1-Diphenylphosphino-2-[α-(N,N-dimethylamino)-o- |
| | diphenylphosphinophenyl) methyl]ferrocene |
| MOD-Mandyphos | 2,2′-(α-N,N-dimethylaminophenylmethyl)-1,1′- |
| | bis[di(3,5-dimethyl-4-methoxyphenyl)phosphino]- |
| | ferrocene |
| (4-CF₃Ph)₂-PtBu₂ | 1-[(2-Di-(4-trifluoromethylphenyl) phosphino) |
| | ferrocenyl]-ethyldi-tert-butylphosphine |
| tBu-Josiphos (PPF-PtBu₂) | [1-[1-[Bis(1,1-dimethylethyl)phosphino]ethyl]-2- |
| | (diphenyl phosphino)ferrocene) |
| MOD-Josiphos | 1-[2-Di-(4-methoxy-3,5-dimethylphenyl) phosphino) |
| | ferrocenyl]ethyldi-tert.-butylphosphine |
| Me-f-Ketalphos | 1,1′-Bis-[3,4-O-isopropylidene-3,4-dihydroxy-2,5- |
| | dimethylphosphanyl]ferrocene |
| DBT-H-SIPHOX | 7-[4,5-Dihydrooxazol-2-yl]-7′-di(3,5-di-tert- |
| | butylphenyl)phosphino-1,1′-spirobiinane |
| DBT-Ph-SIPHOX | 7-[4,5-Dihydro-4-phenyloxazol-2-yl]-7′-di(3,5-di-tert- |
| | butylphenyl)phosphino-1,1′-spirobiinane |
| DBT-Bn-SIPHOX | 7-[4,5-Dihydro-4-benzyloxazol-2-yl]-7′-di(3,5-di-tert- |
| | butylphenyl)phosphino-1,1′-spirobiinane |

### C1. (R) or (S)-2-(3-Chloro-4-methanesulfonyl-phenyl)-3-cyclopentyl-propionic acid Dicyclohexylamine Salt ((R)-4/(S)-4)

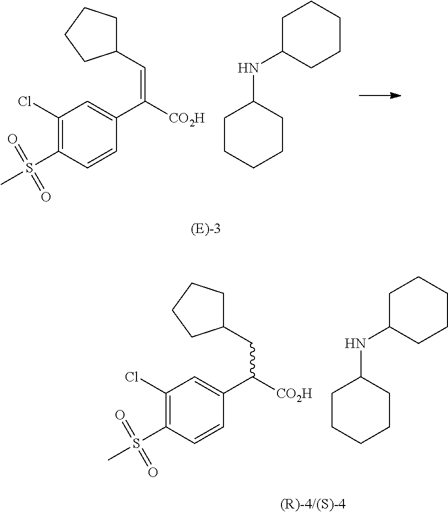

### Ruthenium-Based Catalysts

### Example 1

In a glove box (O₂ content≦2 ppm) a 6 ml autoclave was charged with 0.05 g (0.098 mmol) of (E)-3, 3.58 mg (0.00392 mmol, S/C 25) of [Ru(OAc)₂((S)-3,5-Xyl-MeOBIPHEP)] (catalyst Type VIa/Ru-1) and 1 ml of methanol. The asymmetric hydrogenation was run for 17 h at 50° C. under 50 bar of hydrogen. After cooling to room temperature the pressure was released from the autoclave and the solvent was removed under vacuum to yield (R)-4 in quantitative yield and with 90.0% ee.

¹H-NMR (CDCl₃): δ 8.01 (d, 1H), 7.63 (d, 1H), 7.44 (d, 1H), 3.49 (m, 1H), 3.24 (s, 3H), 2.82 (m, 2H), 2.07 (m, 1H), 1.88 (m, 4H), 1.72 (m, 8H), 1.71-1.57 (m, 5H), 1.46 (m, 2H), 1.33-0.93 (m, 13H); MS m/e (%): 348 ([M₁+NH₄]⁺, 7%); 182 ([M₂+H]⁺, 100%).

### HPLC Method for Determination of Conversion:

Chromolith performance RP-18e 100×4.6 mm, 50% water, 40% acetonitrile and 10% Bu₄NHSO₄ buffer (pH 3), flow 2.0 ml/min, 40° C., 0.0020 ml injection volume, 229 nm. Retention times: unsaturated acid salt 3, 2.0 min, saturated acid salt 4, 2.2 min.

Alternatively, the conversion can be determined with the method for ee determination.

HPLC Method for ee Determination:

Chiralpak-IC column, 250*4.6 mm, 5 μm 80% n-heptane+15% ethanol and 5% of 0.1% trifluoroacetic acid in n-heptane, flow 1.0 ml/min, 30° C., 0.0015 ml injection volume, 210 nm. Retention times: (S)-4 10.6 min, (R)-4 11.3 min.

### Examples 2.1-2.16

In an analogous manner to Example 1 the following hydrogenations were performed at 50° C., under 50 bar of hydrogen (reaction time 16-17 h) using various ruthenium catalysts to afford acid salt 4 in the purity and enantioselectivity indicated in Table 1.

TABLE 1^{A}

| | | | | Salt 4 |
|---|---|---|---|---|
| | | Catalyst | Salt 4 | ee (%)/ |
| Ex. | Catalyst | Type | Purity(%) | Config. |
| 2.1 | Ru(OAc)₂((R)-DiMeOBIPHEP) | VIa/Ru-1 | >99.9 | 87.0/S |
| 2.2 | Ru(OAc)₂((S)-BIPHEMP) | VIa/Ru-1 | >99.9 | 82.6/R |
| 2.3 | Ru(TFA)₂((S)- | VIa/Ru-2 | >99.9 | 88.2/R |
| | TriMeOBIPHEP) | | | |
| 2.4 | [Ru((S)- | VIb/Ru-11 | >99.9 | 78.6/R |
| | MeOBIPHEP)(pCym)I]I | | | |
| 2.5 | [RuH((S)- | VIb/Ru-10 | >99.9 | 75.6/R |
| | BIPHEMP)(C6H6)]BF4 | | | |
| 2.6 | [RuCl₂((S)-3,5-Et- | VIb/Ru-14 | >99.9 | 83.8/R |
| | MeOBIPHEP)(DMF)] | | | |
| 2.7 | Ru(OAc)₂((S)-3,4-Xyl- | VIa/Ru-1 | >99.9 | 82.0/R |
| | MeOBIPHEP) | | | |
| 2.8 | Ru(TFA)₂((S)-(p-Tol- | VIa/Ru-2 | >99.9 | 81.2/R |
| | BIPHEMP) | | | |
| 2.9 | [Ru((R)-BIPHEMP)(p- | VIc/Ru-13 | >99.9 | 73.8/S |
| | Cym)(CH₃CN)](BF₄)₂ | | | |
| 2.10 | Ru(OAc)₂((S)-(6-MeO-2- | VIa/Ru-1 | >99.9 | 81.6/R |
| | Naphthyl)-BIPHEMP) | | | |
| 2.11 | Ru(OAc)₂((R)-SEGPHOS) | VIa/Ru-1 | >99.9 | 76.0/S |
| 2.12 | Ru(OAc)₂((S)-(6-MeO-2- | VIa/Ru-1 | >99.9 | 80.6/R |
| | Naphthyl)-MeOBIPHEP) | | | |
| 2.13 | [Ru((S)- | VIb/Ru-8 | >99.9 | 84.2/R |
| | MeOBIPHEP)(C₆H₆)Br]Br | | | |
| 2.14 | Ru(OAc)₂((S)-(TMBTP) | VIa/Ru-5 | >99.9 | 86.0/R |
| 2.15 | Ru(OAc)₂((S)-(2-Thienyl)- | VIa/Ru-1 | >99.9 | 80.8/R |
| | MeOBIPHEP) | | | |
| 2.16 | Ru(TFA)₂((S)- | VIa/Ru-2 | >99.9 | 87.4/R |
| | TriMeOBIPHEP) | | | |

^{a}Conditions: 6 ml autoclave, 50 mg scale, S/C 25, MeOH (1 ml), 50° C., 50 bar, 16 h.

### Examples 3.1-3.7

In an analogous manner to Example 2 the following hydrogenations were performed at 50° C., under 50 bar of hydrogen (reaction time 16-17 h) and in different solvents using various ruthenium catalysts to afford acid salt 4 in the purity and enantioselectivity indicated in Table 2.

TABLE 2^{a}

| | | | | | Salt 4 |
|---|---|---|---|---|---|
| | | Catalyst | | Salt 4 | ee (%)/ |
| Ex. | Catalyst | Type | Solvent | Purity (%) | Config. |
| 3.1 | [RuI((R,S)- | VIa/Ru-9 | CH₂Cl₂ | >99.9 | 84.0/R |
| | Taniaphos)(2,4- | | | | |
| 3.2 | [Ru((S,R)-(4- | VIb/Ru-12 | CH₂Cl₂ | >99.9 | 88.8/R |
| | CF₃Ph)₂-PtBu₂)(2,4- | | | | |
| 3.3 | [Ru((S,R)-(4- | VIb/Ru-12 | THF | >99 | 80.8/R |
| | CF₃Ph)₂-PtBu₂)(2,4- | | | | |
| 3.4 | [Ru((S,R)-tBu- | VIb/Ru-12 | CH₂Cl₂ | >99.9 | 91.4/R |
| | Josiphos)(2,4-Me- | | | | |
| 3.5 | [Ru((S,R)-tBu- | VIb/Ru-12 | THF | >99.9 | 94.6/R |
| | Josiphos)(2,4-Me- | | | | |
| 3.6 | [Ru((S,R)-tBu- | VIb/Ru-12 | CF₃C₆H₅ | 98 | 94.7/R |
| | Josiphos)(2,4-Me- | | | | |
| 3.7 | [Ru((S,R)-(3,5-Me-4- | VIb/Ru-12 | CH₂Cl₂ | >99.9 | 81.8/R |
| | MeOPh)₂PF- | | | | |

^{a}Conditions: 6 ml autoclave, 50 mg scale, S/C 25, solvent (1 ml), 50° C., 50 bar, 16 h.

### Example 4

In a glove box (O₂ content≦2 ppm) a 185 ml autoclave equipped with a mechanical stirrer was charged with 10.0 g (19.6 mmol) of (E)-3, 1.70 mg (0.00196 mmol, S/C 10000) of [Ru((S,R)-tBu-Josiphos)(2,4-Me-C₅H₅)(CH₃CN)]BF₄ (catalyst Type VIb/Ru-12) and 68 ml of dichloromethane. The autoclave was sealed, pressurized with 50 bar of hydrogen and the asymmetric hydrogenation was run for 17 h at 50° C. After cooling of the autoclave to room temperature, the pressure was released from the autoclave and the thin suspension of (R)-4 was diluted with dichloromethane (30 ml) and sulphuric acid (200 ml, 0.1 mol/l) was added to get a clear colorless solution. The organic phase was separated and washed twice with sulphuric acid (100 ml, 0.2 mol/l), whereas the aqueous phases were washed with dichloromethane (50 ml). The combined organic phases were dried over sodium sulphate, the sodium sulphate was filtered off with suction, a sample for the determination of the conversion (>99.9%) and of the enantioselectivity of the crude product was taken (crude ee=93%) and the solvent was removed under vacuum to give an off-white solid (6.6 g). The crude product was crystallized from 2-propanol to give (R)-8 in 87% yield (5.67 g) and with 98.2% ee.

¹H-NMR (CDCl₃): δ 8.10 (d, 1H), 7.54 (s, 1H), 7.42 (d, 1H), 3.69 (dd, 1H), 3.26 (s, 3H), 2.12 (m, 1H), 2.07 (m, 1H), 1.84 (m, 1H), 1.78 (m, 2H), 1.62 (m, 3H), 1.50 (m, 2H), 1.12 (m, 2H); MS m/e (%): 330 (M⁺, 1%, [M-methylene-cyclopentane]⁺, 100%).

### Example 5

In a glove box (O₂ content≦2 ppm) a 185 ml autoclave equipped with a mechanical stirrer was charged with 10.0 g (19.6 mmol) of (E)-3, 0.85 mg (0.00098 mmol, S/C 20000) of [Ru((S,R)-tBu-Josiphos)(2,4-Me-C₅H₅)(CH₃CN)]BF₄ (catalyst Type VIb/Ru-12) and 68 ml of dichloromethane. The autoclave was sealed, pressurized with 50 bar of hydrogen and the asymmetric hydrogenation was run for 17 h at 50° C. After cooling of the autoclave to room temperature, the pressure was released from the autoclave and the solvent was removed under vacuum to give (R)-4 in quantitative yield and with 92.4% ee.

### Examples 6.1-6.7

In a manner analogous to Example 5 the reactions in Table 3 have been performed using various pressure and various solvents.

TABLE 3

| | | | | Conc. | | | Salt 4 | Salt 4 |
|---|---|---|---|---|---|---|---|---|
| | | | | w/w | | p | Purity | ee (%)/ |
| Ex. | Scale (g) | S/C | Solvent | (%) | Temp. T (° C.) | (bar) | (%) | Config. |
| 6.1^{a} | 1.0 | 1000 | CH₂Cl₂ | 3.6 | 50 | 20 | >99.9 | 92.2/R |
| 6.2^{b} | 0.5 | 1000 | CH₂Cl₂ | 7.0 | 50 | 180 | >99.9 | 90.3/R |
| 6.3^{b} | 0.5 | 1000 | CH₂Cl₂ | 9.7 | 60 | 50 | >99 | 91.8/R |
| 6.4^{a} | 1.0 | 1000 | THF | 5.3 | 50 | 30 | >99 | 95.2/R |
| 6.5^{a} | 1.0 | 1000 | THF | 5.3 | 60 | 40 | >99.9 | 93.6/R |
| 6.6^{b} | 0.3 | 1000 | THF | 5.3 | 40 | 100 | 98 | 94.8/R |
| 6.7^{c} | 10 | 25000 | CH₂Cl₂ | 10.1 | 50 | 70 | >99 | 91.8/R |

^{a}50 ml autoclave.
^{b}35 ml autoclave.
^{c}185 ml autoclave.

### Example 7

### S/C 50000

In a glove box (O2 content≦2 ppm) a 185 ml autoclave equipped with a mechanical stirrer was charged with 10.0 g (19.6 mmol) of (E)-3, 0.36 mg (0.00039 mmol, S/C 50000) of [Ru(OAc)₂((S)-3,5-Xyl-MeOBIPHEP)] (catalyst Type VIa/Ru-1) and 68 ml of methanol. The autoclave was sealed, pressurized with 50 bar of hydrogen and the asymmetric hydrogenation was run for 17 h at 50° C. After liberation of the dicyclohexylamine salt (R)-4 to the free acid (R)-8 with sulphuric acid (0.1 M) and crystallization from 2-propanol, (R)-8 was obtained in 92.5% yield and in 97.0% ee.

### Example 8

### In Situ Preparation of Catalyst

In a glove box (O₂ content≦2 ppm) a 50 ml autoclave was charged with 0.593 mg (0.00078 mmol) [Ru(OAc)₂((S,R)-tBu-Josiphos)] (prepared in situ from [Ru(COD)(OAc)₂] and (S,R)-tBu-Josiphos (S/C 10000, catalyst Type VIb/Ru-4), 4.0 g (7.841 mmol) of (E)-3 and 25 ml of dichloromethane and the hydrogenation was run for 17 h at 50° C. and under 50 bar of hydrogen pressure. The autoclave was cooled to room temperature, the pressure was released and the solvent was removed under vacuum to give (R)-4 in quantitative yield and with 94.3% ee.

### Iridium-Based Catalysts

### Example 9

In a glove box (O₂ content≦2 ppm) 1.94 mg (0.0039 mmol) [Ir(COD)₂]BF₄ and 1.90 mg (0.0043 mmol) (S,S)-(3,5-Xyl-SKEWPHOS) (S/C 25, catalyst Type VIe/Ir-5)) were stirred in dichloromethane (1 ml) in a 6 ml autoclave for 2 h at room temperature. To this resulting solution was added 50 mg (0.098 mmol) of (E)-3 and the hydrogenation was run for 18 h at 50° C. and under 50 bar of hydrogen pressure. The autoclave was cooled to room temperature, the pressure was released and the solvent was removed under vacuum to give (R)-4 in quantitative yield and with 97.0% ee.

### Examples 10.1-10.7

In a manner analogous to Example 9 the reactions in Table 4 have been performed using various Ir-precursors and various chiral phosphine ligands.

TABLE 4^{A}

| | | Catalyst | Salt 4 | Salt 4 ee (%)/ |
|---|---|---|---|---|
| Ex. | Ir-catalyst | Type | Purity (%) | Config. |
| 10.1^{b} | [Ir((S,S)- | VIe/Ir-6 | >99 | 89.6/R |
| | Skewphos(COD)]BARF | | | |
| 10.2^{b} | [Ir(R,S)-MOD- | VIe/Ir-4 | >99 | 90.8/R |
| | Mandyphos(COD)]BARF | | | |
| 10.3^{b} | [Ir((R)-3,5-tBu,4-MeO- | VIe/Ir-2 | >99.9 | 85.8/R |
| | MeOBIPHEP(COD)]BF₄ | | | |
| 10.4^{c} | [Ir((R)-3,5-tBu-MeO- | VIe/Ir-2 | >99.9 | 81.8/R |
| | BIPHEP)(COD)]BF₄ | | | |
| 10.5^{c} | [Ir((S,S)(DBT-H- | VIe/Ir-7 | >99.9 | 90.0/R |
| | SIPHOX)(COD)]BARF | | | |
| 10.6^{c} | [Ir((S,S)(DBT-Ph- | VIe/Ir-7 | >99.9 | 94.7/R |
| | SIPHOX)(COD)]BARF | | | |
| 10.7^{c} | [Ir((S,S)(DBT-Bn- | VIe/Ir-7 | >99.9 | 91.1/R |
| | SIPHOX)(COD)]BARF | | | |
| 10.8^{d} | [Ir((S,S)(DBT-Ph- | VIe/Ir-7 | 98 | 95.8/R |
| | SIPHOX)(COD)]BARF | | | |

^{a}6 ml autoclave, 50 mg substrate, CH₂Cl₂ (1 ml), 50° C., 50 bar, 18 h.
^{b}The complexes have been prepared in situ from [Ir(COD)₂]BARF + 1.1 equiv. diphosphine.
^{c}Isolated complexes were used.
^{d}35 ml autoclave, 0.3 g scale, S/C 1000, MeOH (3 ml), 60° C., 20 bar.

### Example 11

### S/C 500

In a glove box (O₂ content≦2 ppm) 4.95 mg (0.010 mmol) [Ir(COD)₂]BF₄ and 6.08 mg (0.011 mmol) (S,S)-(3,5-Xyl-SKEWPHOS) (S/C 500, catalyst Type VIe/Ir-5) were stirred in dichloromethane (5 ml) for 2 h at room temperature to give a clear, orange-brown solution. This resulting solution was added to a suspension of 2.55 g (5.00 mmol) of (E)-3 and dichloromethane (45 ml) in a 185 ml autoclave equipped with a mechanical stirrer. The asymmetric hydrogenation was run for 18 h at 50° C. under 20 bar of hydrogen. The autoclave was cooled to room temperature, the pressure was released from the autoclave and the solvent was removed under vacuum to give (R)-4 in quantitative yield and with 95.0% ee.

### Examples 12.1-12.4

The reactions in Table 5 have been carried out in a analogous manner to Example 11 but with variable pressure and substrate to catalyst ratio (S/C) and in various solvents,

TABLE 5^{a}

| | | | | | | Salt 4 | Salt 4 |
|---|---|---|---|---|---|---|---|
| | | Scale | | Catalyst | p | Purity | ee (%)/ |
| Ex. | Chiral ligand | (g) | S/C | Type | (bar) | (%) | Config. |
| 12.1^{b} | (R,S)-MOD- | 0.2 | 100 | VIe/Ir-3 | 50 | >99.9 | 88.8/R |
| | Mandyphos | | | | | | |
| 12.2^{b} | (R)-3,5-tBu-4- | 0.2 | 100 | VIe/Ir-2 | 50 | >99.9 | 86.6/R |
| | MeO- | | | | | | |
| | MeOBIPHEP | | | | | | |
| 12.3^{c} | (S,S)- | 2.55 | 500 | VIe/Ir-5 | 20 | >99.9 | 91.0/R |
| | SKEWPHOS | | | | | | |
| 12.4^{c} | (R,S)-MOD- | 2.55 | 500 | VIe/Ir-3 | 20 | 96 | 90.6/R |
| | Mandyphos | | | | | | |

^{a}[Ir(COD)₂]BF₄ was used as iridium precursor. Conditions: the reactions were run in CH₂Cl₂, 17-18 h at 50° C.
^{b}35 ml autoclave, conc. 2.9% w/w.
^{c}50 ml autoclave, conc. 3.7% w/w.

### Example 13

### S/C 2000

In a glove box (O₂ content≦2 ppm) a 185 ml autoclave equipped with a mechanical stirrer was charged with 10.0 g (19.6 mmol) of (E)-3, 9.21 mg (0.0098 mmol, S/C 2000) of [Ir((S,S)-(3,5-Xyl-SKEWPHOS)(COD)]BF₄ (catalyst Type VIe/Ir-5) and 68 ml of dichloromethane. The asymmetric hydrogenation was run for 18 h at 50° C. under 20 bar of hydrogen. After cooling to room temperature the pressure was released from the autoclave and the white suspension was diluted with dichloromethane (45 ml) and hydrochloric acid (200 ml, 0.1 mol/l) was added to get a clear colorless solution. The organic phase was separated and washed twice with hydrochloric acid (100 ml, 0.2 mol/l), whereas the aqueous phases were washed with dichloromethane (50 ml). The combined organic phases were dried over sodium sulphate, the sodium sulphate was filtered off with suction, a sample for the determination of the conversion (>99.9%) and of the enantioselectivity of the crude product was taken (crude ee=95%) and the solvent was removed under vacuum to give a white solid (6.78 g). The crude product was crystallized from 2-propanol to give (R)-8 in 87% yield (5.7 g) and with 99.0% ee.

### Example 14

### In Situ Catalyst Formation from [IrCl(COD)]₂, S/C 2000

In an analogous manner to Example 13 a hydrogenation experiment wherein the catalyst was formed in situ from [IrCl(COD)]₂ and (S,S)-(3,5-Xyl-Skewphos) (catalyst Type VId/Ir-1) was carried out. Crystallization of the crude product from 2-propanol gave (R)-8 in 87% yield (5.67 g) and with 99.1% ee.

### Rhodium-Based Catalysts

### Example 15

In a glove box (O₂ content≦2 ppm) 1.59 mg (0.0039 mmol) [Rh(COD)₂]BF₄ and 1.90 mg (0.0043 mmol) (S,S)-(SKEWPHOS) (S/C 25, catalyst Type VIe/Rh-8) were stirred in methanol (1 ml) for 90 min at room temperature to give a clear, orange-brown solution. To this resulting solution was added in a 6 ml autoclave 0.05 g (0.098 mmol) of (E)-3. The asymmetric hydrogenation was run for 18 h at 50° C. under 50 bar of hydrogen. The autoclave was cooled to room temperature, the pressure was released from the autoclave and the solvent was removed under vacuum to give (R)-4 in quantitative yield and with 85.4% ee.

### Examples 16.1-16.13

The reactions in Table 6 have been performed in a analogous manner to Example 15 but with different chiral ligands, Rh precursors and in various solvents.

TABLE 6^{a}

| | | | | Salt 4 | Salt 4 |
|---|---|---|---|---|---|
| | | Catalyst | | Purity | ee (%)/ |
| Ex. | Chiral ligand | Type | Solvent | (%) | Config. |
| 16.1 | (S,S)-SKEWPHOS | VIe/Rh-8 | THF | >99.9 | 87.2/R |
| 16.2 | (S,S)-SKEWPHOS | VIe/Rh-8 | EtOH | >99 | 83.2/R |
| 16.3 | (S,S)-(3,5-Xyl- | VIe/Rh-8 | THF | >99 | 87.8/R |
| | Skewphos) | | | | |
| 16.4^{b} | (R,S)-MOD- | VIe/Rh-5 | MeOH | >99 | 90.4/R |
| | Mandyphos | | | | |
| 16.5^{b} | (R,S)-MOD- | VIe/Rh-5 | EtOH | >99 | 90.4/R |
| | Mandyphos | | | | |
| 16.6^{c} | (R,S)-MOD- | VIe/Rh-5 | CH₂Cl₂ | >99.9 | 91.8/R |
| | Mandyphos | | | | |
| 16.7^{d} | (R,S)-MOD- | VIe/Rh-6 | THF | >99 | 88.2/R |
| | Mandyphos | | | | |
| 16.8^{b,e} | (R,S)-MOD- | VIe/Rh-7 | CH₂Cl₂ | >99.9 | 87.2/R |
| | Mandyphos | | | | |
| 16.9^{b,f} | (R,S)-MOD- | VId/Rh-2 | CH₂Cl₂ | >99.9 | 88.0/R |
| | Mandyphos | | | | |
| 16.10^{b,g} | (R,S)-MOD- | VId/Rh-1 | CH₂Cl₂ | >99.9 | 86.8/R |
| | Mandyphos | | | | |
| 16.11^{b,h} | (R,S)-MOD- | VId/Rh-3 | CH₂Cl₂ | >99.9 | 87.2/R |
| | Mandyphos | | | | |

^{a}Conditions: 6 ml autoclave, S/C 25, [Rh(COD)₂]BF₄ as Rh-precursor, 50 mg scale, solvent (1 ml), 50 bar, 50° C., 18 h.
^{b}35 ml autoclave, 0.2 g scale, S/C 100.
^{c}50 ml autoclave, 1.0 g scale, S/C 200, 20 bar.
^{d}Precursor: [Rh(COD)₂]SbF₆.
^{e}Precursor: [Rh(COD)₂]OTf.
^{f}Precursor: [Rh(TFA)(COD)]₂.
^{g}Precursor: [Rh(Cl)(COD)]₂.
^{h}Precursor: [Rh(acac)(COD)]₂.

### C2. (R)-2-(3-Chloro-4-methanesulfonyl-phenyl)-3-cyclopentyl-propionic acid Sodium Salt ((R)-5)

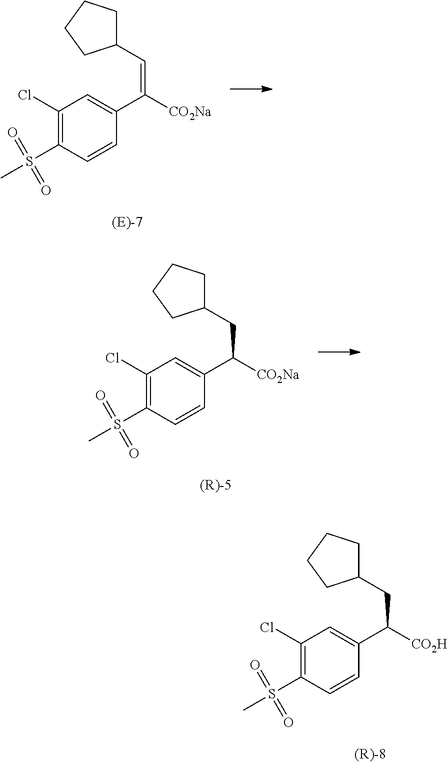

### Ruthenium-Based Catalysts

### Example 17

In a glove box (O₂ content≦2 ppm) a 6 ml autoclave was charged with 4.94 mg (0.0057 mmol, S/C 25) [Ru((S,R)-tBu-Josiphos)(2,4-Me-C₅H₅)(CH₃CN)]BF₄ (catalyst Type VIb/Ru-12), 0.05 g (0.143 mmol) of (E)-7 and 1 ml of THF. The asymmetric hydrogenation was run for 17 h at 50° C. under 50 bar of hydrogen. The autoclave was cooled to room temperature, the pressure was released from the autoclave and the solvent was removed under vacuum to give (R)-5 in quantitative yield and with 92.4% ee.

### Example 18.1-18.2

The reactions in Table 7 have been carried out in an analogous manner to Example 17 but in methanol as the solvent.

TABLE 7^{A}

| | | Catalyst | Salt 5 | Salt 5 ee (%)/ |
|---|---|---|---|---|
| Ex. | Catalyst | Type | Purity (%) | Config. |
| 18.1 | [Ru(OAc)₂(S)-3,5-Xyl- | VIa/Ru-1 | >99.9 | 89.8/R |
| | MeO-BIPHEP] | | | |
| 18.2 | [Ru(OAc)₂((S)- | VIa/Ru-5 | >99.9 | 88.0/R |
| | TMBTP)] | | | |

^{a}Conditions: 6 ml autoclave, 50 mg scale, S/C 25, MeOH (1 ml, conc. 5.9% w/w), 50° C., 50 bar, 17 h.

### Example 19

### In Situ Preparation of Catalyst

In a glove box (O₂ content≦2 ppm) a 35 ml autoclave with a 15 ml glass insert was charged with 0.67 mg (0.00086 mmol) [Ru(OAc)₂((S,S,S,S)-Me-f-KetalPhos) (prepared in situ from [Ru(COD)(OAc)₂] and (S,S,S,S)-Me-f-KetalPhos) (S/C 1000, catalyst Type VIa/Ru-7), 0.3 g (0.855 mmol) of (E)-7 and 5 ml of THF and the hydrogenation was run for 17 h at 50° C. and under 50 bar of hydrogen pressure. The autoclave was cooled to room temperature, the pressure was released and the solvent was removed under vacuum to give (R)-5 in quantitative yield and with 92.5% ee.

### Iridium-Based Catalysts

### Example 20

### S/C 500

In a glove box (O₂ content≦2 ppm) (E)-7 (1.40 g, 4.00 mmol) was treated with dichloromethane (10 ml) and water (2 ml) to give a bi-phasic mixture. To this bi-phasic mixture was added [Ir((S,S)-(3,5-Xyl-SKEWPHOS))(COD)] (7.52 mg, 0.008 mmol, S/C 500, catalyst Type VIe/Ir-5) in 8 ml dichloromethane. The autoclave was sealed, pressurized with 20 bar of hydrogen and the hydrogenation was run for 18 h at 50° C. After cooling of the autoclave to 20° C., the pressure was released, the reaction mixture was transferred into a separation funnel and the autoclave was rinsed with water (6 ml). The organic phase was separated and the water phase was washed with dichlormethane (5 ml). The water phase was treated with hydrochloric acid (aq., 1 mol/1, 4.5 ml) resulting in a pH of ca. 1 and the resulting milky suspension was washed twice with dichloromethane (in total 30 ml) and the organic phase was washed with water (10 ml). The volume of the combined organic phases was reduced under vacuum and the resulting oil was dried under vacuum to weight constancy to give (R)-8 in the form of a colorless oil, which solidified upon standing, in 96.1% yield and 95% ee.

### Example 21

### Re-Use of Catalyst, Catalyst Type VIe/Ir-5

In an analogous experiment to Example 20 but on 3 g scale (8.552 mmol) and at S/C 1000 (185 ml autoclave with glass insert) within a reaction time of 6 h full conversion was achieved. The autoclave was opened in a glove box (O₂ content≦2 ppm) the water phase was removed and the autoclave was charged with the organic phase from the first run, (E)-7 (3 g) water (4.5 ml) and 0.8 mg [Ir((S,S)-(3,5-Xyl-SKEWPHOS))(COD)] (0.000855 mmol, 10% of the original catalyst loading). The hydrogenation was run for 16 h for the second and third run and 24 h for the forth run (4^{th} run with 20% of additional catalyst), respectively. In this manner the catalyst has been re-used 3 times affording quantitative yield of (R)-5 and 96.0% ee. This corresponds to a S/C ratio of 2858.

### Example 22.1-22.2

In a glove box (O₂ content≦2 ppm) a 35 ml autoclave with a 15 ml glass insert was charged with 6.52 mg (0.005 mmol) [Ir(COD)((R)-3,5-iPr-MeOBIPHEP)]BF₄ (prepared in situ from [Ir(COD)₂]BF₄ and (R)-3,5-iPr-MeOBIPHEP) (S/C 20, catalyst Type VIe/Ir-2), 0.05 g (0.100 mmol) of (E)-7 and 1 ml of CH₂Cl₂/PEG (1:1) and the hydrogenation was run for 17 h at 50° C. and under 20 bar of hydrogen pressure to give (R)-5 in quantitative yield and with 83.2% ee.

### Example 23.1-23.3

In an analogous manner to Example 22 but in various solvents and at various S/C the reactions in Table 8 have been performed using [Ir((S,S)-3,5-Xyl-Skewphos)(COD)]BF₄ (catalyst Type VIe/Ir-5) as the catalyst.

TABLE 8

| | | | Salt 5 | Salt 5 |
|---|---|---|---|---|
| | | Solvent; Conc. | Purity | ee (%)/ |
| Ex. | S/C | (w/w %) | (%) | Config. |
| 23.1^{a} | 500 | CH₂Cl₂/propylene | 98 | 94.6/R |
| | | carbonate (95:5); | | |
| | | 5.1 | | |
| 23.2^{a} | 500 | CH₂Cl₂/ethylene | 98.6 | 93.8/R |
| | | glycol (95:5); 5.1 | | |
| 23.3^{a} | 1000 | CH₂Cl₂/H₂O | 98 | 94.2/R |
| | | (90:10); 5.1 | | |

^{a}1.4 g (4.0 mmol) substrate, 50 ml autoclave, solvent (20 ml), 16-19 h.

### C3. (R)-2-(3-Chloro-4-methanesulfonyl-phenyl)-3-cyclopentyl-propionic acid ((R)-8)

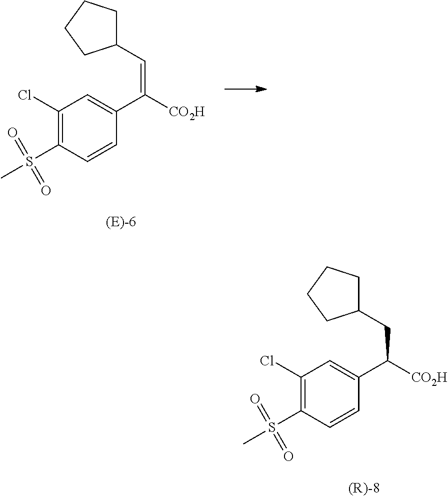

### Example 24

### S/C 500

In a glove box (O₂ content≦2 ppm) a 35 ml autoclave equipped with a magnetic stirrer was charged with 0.2 g (0.608 mmol) of (E)-6, 1.11 mg (0.00122 mmol, S/C 500) of [Ru((S)-(3,5-Xyl-MeOBIPHEP)] (Catalyst Type VIa/Ru-1) and 4 ml of methanol. The asymmetric hydrogenation was run for 17 h at 50° C. under 50 bar of hydrogen. After cooling to room temperature the pressure was released from the autoclave, a sample for analytics (conversion and enantioselectivity) was taken and the solvent was removed under vacuum to give (R)-8 as an off-white solid in quantitative yield and with 87.2% ee.

### Example 25

### S/C 1000

In a glove box (O₂ content≦2 ppm) a 35 ml autoclave equipped with a magnetic stirrer was charged with 0.3 g (0.912 mmol) of (E)-6, 0.79 mg (0.00091 mmol, S/C 1000) of [Ru((S,R)-tBu-Josiphos)(2,4-Me-C₅H₅)(CH₃CN)]BF₄ (Catalyst Type VIa/Ru-4) and 6 ml of a dichloromethane/water (9:1) mixture. The asymmetric hydrogenation was run for 17 h at 50° C. under 50 bar of hydrogen. After cooling to room temperature the pressure was released from the autoclave, a sample for analytics (conversion and enantioselectivity) was taken and the solvent was removed under vacuum to give (R)-8 as an off-white solid in quantitative yield and with 94.4% ee.

1. A process for the preparation of a (R)-2-phenyl propionic acid derivative of formula I, 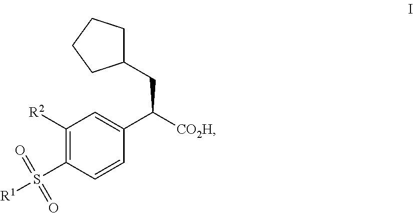
or a salt thereof, wherein R¹ is C₁₋₆-alkyl and R² is hydrogen or halogen, comprising one or more of the following steps:
a) the oxidation of a sulfide of formula II, 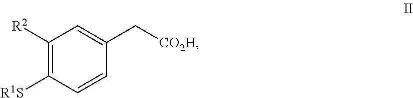
wherein R¹ and R² are as defined above, with performic acid to form a sulfone of the formula III, 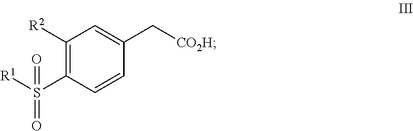
b) the conversion of the sulfone of formula III with cyclopentane carbaldehyde and acetic anhydride in the presence of a base to form an acrylic acid derivative of formula IV, 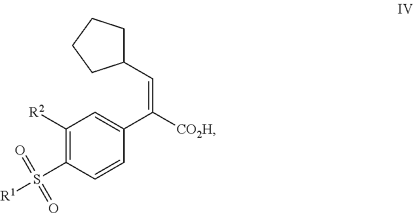
or a salt thereof, wherein R¹ and R² are as defined above; and
c) the asymmetric hydrogenation on the acrylic acid derivative of formula IV, or of a salt thereof, in the presence of a complex catalyst to form the propionic acid derivative of formula I, or of a salt thereof.

2. The process of claim 1, wherein the performic acid used for the oxidation in step a) is produced in situ by adding hydrogen peroxide to formic acid at a temperature of 20° C. to 60° C.

3. The process of claim 1, wherein the base used in step b) is selected from an alkali acetate.

4. The process of claim 1, wherein the conversion in step b) is performed in the presence of an organic solvent at a reaction temperature of from 20° C. to 100° C.

5. The process of claim 1, wherein the complex catalyst used for the asymmetric hydrogenation in step c) is selected from the group consisting of
Ru(Z)₂D, [Ru(Z)₂₋ₚ(D)(L)ₘ](Y)ₚ, [Ru(D)(L)₂](Y)₂,
[M(D)LX], and [M(D)L]⁺Y⁻,
wherein
each Z is independently selected from the group consisting of hydrogen, halogen, η⁵-2,4-pentadienyl, η⁵-2,4-dimethyl-pentadienyl and the group A-COO⁻ wherein
A is selected from the group consisting of C₁₋₆-alkyl, aryl, halogenated C₁₋₆-alkyl and halogenated aryl;
Y is a non-coordinating anion;
D is a chiral phosphine ligand;
L is a neutral ligand;
M is Iridium or Rhodium
X is a halogen atom;
m is an integer from 1 to 3; and
p is 1 or 2.

6. The process of claim 5, wherein the phosphine ligand D is selected from the group consisting of: 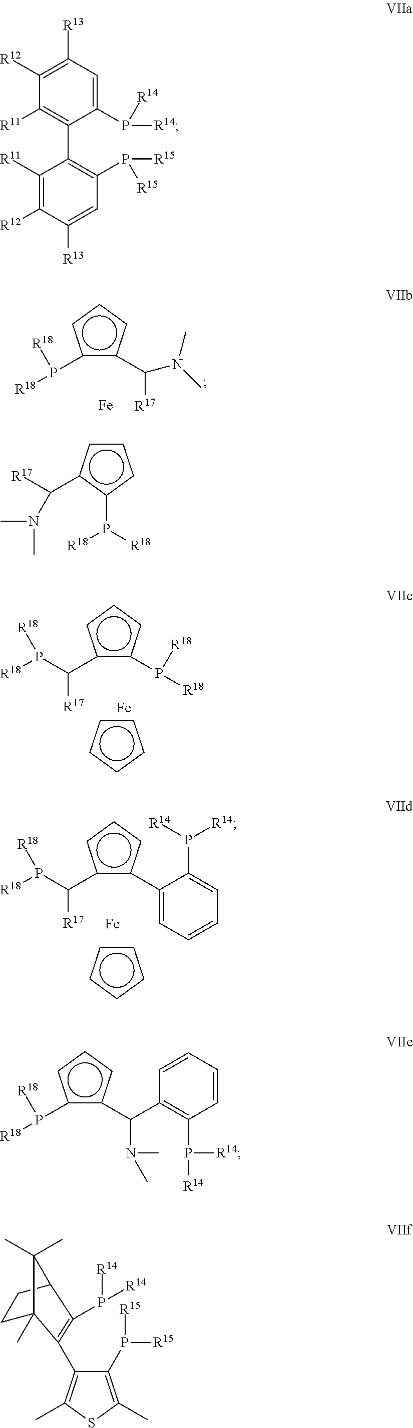 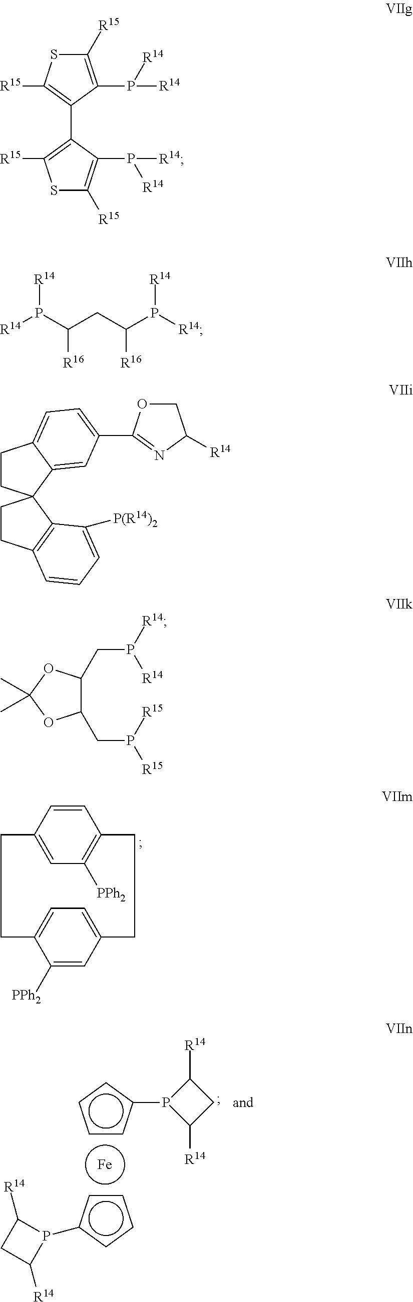 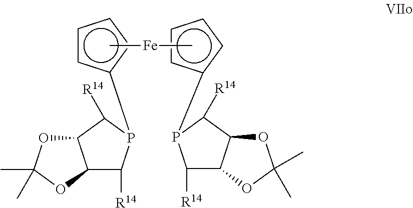
wherein
R¹¹ is selected from the group consisting of C₁₋₆-alkyl, C₁₋₆-alkoxy, hydroxy and C₁₋₆-alkyl carbonyl oxy;
R¹² and R¹³ independently of each other are selected from the group consisting of hydrogen, C₁₋₆-alkyl, C₁₋₆-alkoxy and di-(C₁₋₆-alkyl)amino; or
R¹¹ and R¹² which are attached to the same phenyl group or
R¹² and R¹³ which are attached to the same phenyl group, taken together, are —X—(CH₂)ᵣ—Y—,
wherein X is —O— or —C(O)O—, Y is —O— or —N(C₁₋₆-alkyl)- and r is an integer from 1 to 6, or a CF₂ group,
or both R¹¹s, taken together, are —O—(CH₂)ᵣ—O— or O—CH(CH₃)—(CH₂)ᵣ—CH(CH₃)—O—, wherein r is an integer from 1 to 6, or
R¹¹ and R¹², or R¹² and R¹³, together with the carbon atoms to which they are attached, form a naphthyl, tetrahydronaphthyl or dibenzofuran ring;
R¹⁴ and R¹⁵ independently of each other are selected from the group consisting of C₁₋₆-alkyl, C₃₋₈-cycloalkyl, phenyl, napthyl and heteroaryl, substituted with 0 to 7 substituents independently selected from the group consisting of C₁₋₆-alkyl, C₁₋₆-alkoxy, di(C₁₋₆-alkyl)amino, morpholino, phenyl and tri(C₁₋₆-alkyl)silyl, carboxy, and C₁₋₆-alkoxycarbonyl;
R¹⁶ is C₁₋₆-alkyl;
R¹⁷ is C₁₋₆-alkyl; and
R¹⁸ is selected from the group consisting of aryl, heteroaryl, C₃₋₈-cycloalkyl and C₁₋₆-alkyl.

7. The process of claim 6, wherein the phosphine ligand D is selected from the group consisting of compounds of formulas VIIa, VIIc, VIIh, VIIi and VIIo.

8. The process of claim 5, wherein Y is selected from the group consisting of halides, AsF₆⁻, BF₄⁻, ClO₄⁻, SbF₆⁻, PF₆⁻, B(phenyl)₄⁻, B(3,5-di-trifluoromethyl-phenyl)₄⁻, CF₃SO₃⁻, and C₆H₅SO₃⁻.

9. The process of claim 5, wherein L is selected from the group consisting of ethylene, propylene, cyclooctene, 1,3-hexadiene, 1,5-hexadiene, bicyclo-[2.2.1]hepta-2,5-diene, (Z,Z)-1,5-cyclooctadiene, benzene, hexamethylbenzene, 1,3,5-trimethylbenzene, p-cymene and solvents selected from the group consisting of tetrahydrofuran, N,N-dimethylformamide, acetonitrile, dimethylsulfoxide, benzonitrile, acetone, methanol and pyridine.

10. The process of claim 5, wherein the complex catalyst is selected from:
catalyst type VIa/Ru-1; catalyst type VIa/Ru-2; catalyst type VIa/Ru-3; catalyst type VIa/Ru-4; catalyst type VIa/Ru-5; catalyst type VIa/Ru-6; catalyst type VIa/Ru-7; catalyst type VIb/Ru-8; catalyst type VIa/Ru-9; catalyst type VIb/Ru-10; catalyst type VIb/Ru-11; catalyst type VIb/Ru-12; catalyst type VIc/Ru-13; catalyst type VIb/Ru-14; catalyst type VId/Ir-1; catalyst type VId/Rh-1; catalyst type VId/Rh-2; catalyst type VId/Rh-3; catalyst type VIe/Ir-2, catalyst type VIe/Ir-3; catalyst type VIe/Ir-4; catalyst type VIe/Ir-5; catalyst type VIe/Ir-6; catalyst type VIe/Ir-7; catalyst type VIe/Rh-4; catalyst type VIe/Rh-5; catalyst type VIe/Rh-6; catalyst type VIe/Rh-7); and catalyst type VIe/Rh-8.

11. The process of claim 1, wherein the asymmetric hydrogenation in step c) is performed in an organic solvent at a reaction temperature between 10° C. and 100° C. and a pressure between 1 and 180 bar.

12. The process of claim 1, wherein the acrylic acid derivative of the formula IV used for the asymmetric hydrogenation in step c) is selected from the free acid, the dicyclohexylamine salt or from an alkali metal salt thereof.

13. The process of claim 1, wherein the substituents of the double bond in the acrylic acid derivative of the formula IV have an (E)-configuration.

14. A process for the preparation of a compound of the general formula Xa, 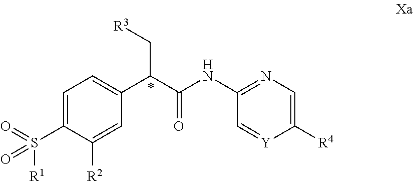
wherein the process comprises the process steps as defined in claim 1.

15. The process of claim 14, wherein the compound of general formula Xa is a compound of the formula Xb, [chemical structure]
